(19) United States
(12) Patent Application Publication
(10) Pub. No.: US 2009/0171326 A1
(43) Pub. Date: Jul. 2, 2009

(54) Medical Device and Method

(75) Inventors: Kristian Hohla, Vatertetten (DE); Stephen Slade, Houston, TX (US)

Correspondence Address: SMITH FROHWEIN TEMPEL GREENLEE BLAHA, LLC, Two Ravinia Drive, Suite 700, ATLANTA, GA, 30346, US

(73) Assignee: SK TECHNOLOGIES GMBH, Munchen (DE)

(21) Appl. No.: 11/994,236

(22) PCT Filed: Jun. 9, 2006
(86) PCT No.: PCT/EP06/63054

§ 371 Date: Jan. 8, 2009

Related U.S. Application Data
(60) Provisional application No. 60/695,591, filed on Jun. 29, 2005.
(60) Provisional application No. 60/718,074, filed on Sep. 16, 2005.
(60) Provisional application No. 60/727,744, filed on Oct. 18, 2005.

(51) Int. Cl. A61B 18/18

(52) U.S. Cl. 606/6

(57) ABSTRACT

A medical device comprising a lens epithelial cell deactivation unit adapted for selectively deactivating lens epithelial cells of an eye.

The invention relates to a medical device.

The invention further relates to a medical method.

A cataract is an opacity which develops in the crystalline lens of the eye or in its envelope. Cataracts form for a variety of reasons, including long term ultraviolet exposure, secondary effects of diseases such as diabetes, or simply due to advanced age. They are usually a result of denaturation of lens proteins. Genetic factors are often a cause of congenital cataracts and may also play a role in predisposing someone to cataracts. Cataracts may also be produced by eye injury or physical trauma.

A common treatment is cataract surgery to remove the cloudy lens. There are two types of surgery that can be used to remove cataracts, extra-capsular and intra-capsular surgery. Extra-capsular surgery consists of removing the lens but leaving the majority of the lens capsule intact. Ultra-High frequency sound waves (phacoemulsification) are sometimes used to break up the lens before extraction. Intra-capsular surgery involves removing the entire lens of the eye, including the lens capsule, but it is rarely performed in modern practice. In either extra-capsular surgery or intra-capsular surgery, the lens is replaced with a plastic lens (an intraocular lens implant) which remains permanently in the eye.

Christopher P. Born, M. D. et al., Effect of intraocular lens optic design on posterior capsular opacification, J Cataract Refract Surg, vol. 16, March 1990, pp. 188-192, R. Bougaran et al., A Capsular Ring to Prevent Complications After Cataract Surgery, Investigative Ophthalmology & Visual Science, vol. 38, No. 4, Mar. 15, 1997, p. S144, David A. Hiles et al., Modern Intraocular lens implants in children with new age limitations, J. Cataract Refract Surg, vol. 13, September 1987, pp. 493-497, Barry A. Maltzman, M. D. et al., Effect of the laser ridge on posterior capsule opacification. J. Cataract Refract Surg, vol. 15, November 1989, pp. 644-646, T. Nagamoto et al., Prevention of Secondary Cataract by Blocking Capsular Adhesion with a Newly Designed Intraocular Lens, IOVS, vol. 40, No. 4, Mar. 15, 1999, p. S295, Toyofumi Nagata et al., Optic Sharp Edge or Convexity: Comparison of Effects on Posterior Capsular Opacification, Jpn J Opthalmol, vol. 40, 1996, pp. 397-403, Okihiro Nishi, M. D. et al., Inhibition of Migrating Lens Epithelial Cells at the Capsular Bend Created by the Rectangular Optic Edge of a Posterior Chamber Intraocular Lens, Opthalmic Surgery and Lasers, vol. 29, No. 7, July 1998, pp. 587-594, Tetsuro Oshika, M. D. et al., Two year clinical study of a soft acrylic intraocular lens, J. Cataract Refract Surg, vol. 22, January/February 1996, pp. 104-109, Keiko Yamada, M.D., et al., Effect of intraocular lens design on posterior capsule opacification after continuous curvilinear capsulorhexis, J. Cataract Refract Surg, vol. 21, November 1995, pp. 697-700, Morcher—Special Implants, www.morcher.com/produkte/sonder/engl/e.sub.-sonder.htm, Sep. 17, 1999, pp. 1-4, Beatrice Cochener, M. D. et al., Secondary cataract inhibition by a capsular ring-bound sustained drug delivery system, Best papers from the 1998 ASCRS meeting, p. 7, Sean Charles, PCO Delayed in Paediatric Cataract Patients With Foldable Acrylic IOL, Cataract & Refractive Surgery Euro Times, May-June 1999, p. 10 disclose methods related to cataract surgery. Usha P. Andley, Jennifer G. Weber, “Ultraviolet Action Spectra for Photobiological Effects in Cultured Human Lens Epithelial Cells”, Photochemistry and Photobiology, Vol. 62, No. 5, pp. 840 to 846, 1995 discloses an action spectrum in cultured HLE cells. Joussen, A. M. et al., Low-Dose Rate Ionizing Irradiation for Inhibition of Secondary Cataract Formation, Int. J. Radiation Oncology Biol. Phys., Vol. 49, No. 3, pp. 817 to 825, 2001 discloses inhibition of lens epithelial cell proliferation with a capsular bag ring, labeled with a β-emitting radioisotope.

It is an object of the invention to allow for an efficient treatment of an eye suffering of a cataract.

In order to achieve the object defined above, a medical device and a medical method according to the independent claims are provided.

According to an exemplary embodiment of the invention, a medical device is provided, the medical device comprising a lens epithelial cell deactivation unit adapted for deactivating lens epithelial cells of an eye, particularly using electromagnetic radiation.

According to another exemplary embodiment of the invention, a medical method is provided, the medical method comprising deactivating lens epithelial cells of an eye, particularly using electromagnetic radiation.

Selectively deactivating lens epithelial cells of an eye, preferably without harming or injuring other components of the eye, may allow for an efficient prevention of the so-called second cataract. Such a selective deactivation of lens epithelial cells may be achieved, for instance, by irradiating the lens epithelial cells with UV radiation which deactivates the lens epithelial cells but does not destroy other components of the eye. More generally, such a selective deactivation may be any kind of treatment which inhibits the activity of the lens epithelial cells, but does not inhibit the activity of other components of the eye. The treatment with electromagnetic radiation may be combined as well by a local treatment of the lens epithelial cells with radioactivity or chemicals.

According to an exemplary embodiment of the invention, an apparatus is provided which is capable of selectively destroying, deactivating or removing lens epithelial cells of an eye. By taking this measure, in particular after having removed lens material in the context of a conventional cataract surgery procedure, it may become possible to prevent or suppress growth of new lens cells initiated by the lens epithelial cells, which would have the undesired consequence that similar symptoms would arise again. This may prevent a secondary cataract.

For instance, a beam of ultraviolet radiation may be caused to propagate from an UV light source, e.g. a lamp or a laser, through an optical fiber or other optical elements into the lens (emptied by removing the lens filling material beforehand) in order to selectively interact with the lens epithelial cells to kill them. During such a procedure, the lens (which can be assumed to have a structure similar like a grape) may be filled with saline solution or with a gas before the ultraviolet radiation is guided to the lens epithelial cells located in the front portion of the skin of the grape-like lens.

The electromagnetic radiation source may be adapted for generating the electromagnetic radiation when being located within a lens capsule of the eye. It is possible that the UV radiation for deactivating lens epithelial cells is generated directly in the lens capsule. For instance, it is possible to induce emission of UV radiation by igniting a plasma using a laser directly in the lens capsule. Such a laser beam can be irradiated during the procedure of emptying the lens capsule, that is to say when removing the lens material from the lens capsule.

Thus, an arrangement for the surgical treatment of cataract in a single surgical procedure may be made possible. An opaqueness of the lens may be conventionally removed by substituting the lens material by other material. However, in order to avoid re-growth of lens cells in the remaining cell tissue, an optical fiber may supply electromagnetic radiation of a particular wavelength to the capsule in order to deactivate the lens epithelial cells. By means of a (for instance multi-) cannula, material may be suctioned out of the eye or may be selectively inserted into the empty lens capsule. Such material may be used for cleaning or rinsing, may be used for generating a desired shape of the lens filled with the material, and may be used to fill the empty lens capsule with material (gas or fluid) which is not very prone for ultraviolet radiation absorption and thus does not disturb or weaken considerably the epithelial cell deactivation process.

Optionally, a collar or the like may be used in order to separate the lens capsule of an anterior eye portion.

Next, further exemplary embodiments of the invention will be explained.

In the following, further exemplary embodiments of the medical device will be explained. However, these embodiments also apply for the medical method.

The lens epithelial cell deactivation unit may comprise an electromagnetic radiation source adapted to emit radiation for deactivating lens epithelial cells of an eye. Such a radiation source may be an ultraviolet lamp, laser, for instance an UV laser or a laser diode. Such an electromagnetic radiation source may be adapted to generate electromagnetic radiation of sufficient intensity and of a proper wavelength capable of destroying selectively the lens epithelial cells, whereas the remaining portions of the eye should be essentially uninfluenced by such a procedure.

The electromagnetic radiation source may be adapted to emit ultraviolet radiation for deactivating lens epithelial cells of an eye. From physical reasons it could be between 100 nm and 250 nm, preferably 180 nm and 230 nm. more preferably between 190 nm- and 210 nm. It is believed that especially this wavelength region, particularly in the range between essentially 190 nm and essentially 230 nm, is particularly advantageous for obtaining an efficient deactivation selectively of lens epithelial cells, since this frequency range is powerful enough to reliably destroy lens epithelial cells, but to prevent deterioration of other parts of the eye. A range between 297 nm and 302 nm might be appropriate for the purpose of the invention.

The electromagnetic radiation source may be adapted to emit electromagnetic radiation in such a manner that at least one of the group consisting of one or more wavelengths to be applied sequentially or simultaneously, an intensity, and a duration of the emission of electromagnetic radiation is controllable. These parameters may be used as fit parameters to properly adjust an UV lens irradiation therapy for treating cataract. For instance, a user interface controllable by a physician/surgeon may be provided which may allow to accurately design or define various parameters of the electromagnetic radiation pattern. By taking this measure, individual circumstances of a surgery procedure may be taken into account. An efficient removal or deactivation of lens epithelial cells may include a continuous or pulsed application of UV light of one or a plurality of different frequencies in a defined order, with defined intensities.

The lens epithelial cell deactivation unit may comprise an optical fiber coupled to the electromagnetic radiation source, the optical fiber being adapted for guiding electromagnetic radiation emitted by the electromagnetic radiation source to lens epithelial cells of an eye. An optical fiber may be a transparent optical fiber, usually made of glass or fused silica or plastic, for transmitting light. An optical fiber is a cylindrical dielectric waveguide that transmits light along its axis, by the process of total internal reflection. The fiber consists of a core surrounded by a cladding layer. For the fiber to guide the optical signal the refractive index of the core must be greater than that of the cladding so that light can be confined to the guiding layer by total internal reflection. The boundary between the core and cladding may either be abrupt, in step-index fiber, or gradual, in graded-index fiber. Glass optical fibers may be made from silica, but some other materials, such as fluorozirconate, fluoroaluminate, and chalcogenide glasses may be used. Typically the difference between core and cladding is less than one percent.

By using an optical fiber, the locations of the electromagnetic source on the one hand and the eye on the other hand may be separated, so that essentially no conditions have to be considered when designing the electromagnetic radiation source. At an end tip of the optical fiber, the electromagnetic radiation, for instance ultraviolet radiation, may emerge and may be selectively directed to a desired portion of the eye. Since a optical fiber may be stable and flexible, a physician holding such a optical fiber may accurately define the location to which the electromagnetic radiation shall be directed.

A hollow waveguide filled with gas and/or liquid and/or a rinsing fluid may be used as a substitution for a conventional optical fiber.

The device may comprise a lens material removal unit adapted for removing lens material from an inside of a lens capsule of the eye. In other words, before, after or simultaneously with the deactivation of the lens epithelial cells, the lens material may be removed to treat cataract and to remove opaqueness. Both, removal of the lens material and deactivation of the lens epithelial cells may be performed with one and the same device which may make the surgical procedure easy and fast.

Particularly, the lens material removal unit may comprise a suction unit for sucking off lens material from an inside of a lens capsule of an eye. Such a suction unit may be a cannula to which a low pressure (or negative pressure) may be applied by an external pump. Under the influence of such a low pressure, the lens material may simply be suctioned out of the lens.

The lens material removal unit may comprise an ultrasound source unit for generating ultrasound so as to promote the removal of lens material from an inside of a lens capsule of an eye. Those skilled in the art will recognize that such an ultrasound source unit may be similar or equal to an ultrasound source unit employed conventionally for promoting removal of lens material from an inside of a lens for treating cataract.

The lens epithelial cell deactivation unit and the lens material removal unit may be integrally formed in a common shared device. By taking such a measure, it may be prevented that two different apparatuses need to be for treating the “first” cataract and the “second” cataract. In contrast to this, both such deficiencies may be treated in a common surgical procedure.

The device may comprise a cannula adapted to a supply a lens capsule of an eye with a fluid. For instance, fluids for rinsing, filling the empty lens, etc. may be selectively supplied to the interior of the lens so that no modification of the apparatus is necessary for rinsing, removing lens cell material or filling the lens with such material. Using valves or the like may allow to connect the cannula with different fluid containers so as to selectively allow to use a cannula for providing different liquids/gases or for removing material from the inside of the lens capsule and to transport such material into a waste container.

The cannula may be adapted to supply a lens capsule of an eye with at least one of the group consisting of a liquid and a gas. For instance, a liquid (in which solid particles like salt may be dissolved) or a gas (for instance air) may be filled into the lens capsule in order to adjust accurately the electromagnetic radiation absorption properties and the external geometrical shape of the empty lens skin, or for rinsing.

The cannula may comprise a plurality of separate cannula channels, each of the cannula channels being adapted for separately and selectively supplying a lens capsule of an eye with a fluid. Thus, the different cannula channels may be provided for inserting material into or for removing material from the lens capsule. It is also possible to provide different cannula channels for different liquids to be inserted into the lens capsule or for inserting different gases into the lens capsule. This may provide for a flexible and high performance system. It is even possible to use a liquid as a light guide.

The cannula and at least one of the group consisting of the lens epithelial cell deactivation unit and the lens material removal unit may be integrally formed in a common device. More particularly, the cannula, the lens epithelial cell deactivation unit and the lens material removal unit may all be integrally formed in a common device. This may allow for a miniature construction and for high performance of the miniature device.

A cannula control unit may be provided to control a fluid flow through the cannula. Such a control unit may be realized as a user input/output unit or a user interface. It may also be realized as a console or panel or desk switchboard. This may allow a physician, even during using her or his hands when performing the surgical steps, to accurately control the functionality of the cannula.

The cannula control unit may be adapted to control at least one of a fluid flow rate and a kind of fluid to flow through the cannula. Even concentrations, temperatures, mixture ratios or other parameters characterizing the fluid to flow through the cannula may be adjusted.

The device may further comprise a cannula adapted to suck off a fluid from an inside of a lens capsule of an eye. This further cannula may be provided to remove material from inside the lens capsule, for instance by applying a negative pressure or a vacuum. Also an ultrasound may be used to support or promote removal of the material from the lens to the exterior of the eye.

The further cannula and the suction unit may be formed as a common device. This may allow for a very simple construction of the entire system.

The further cannula and the cannula may be formed as a common device. Therefore, one and the same cannula may be used for inserting fluid into the lens capsule and for removing fluid from the lens capsule to an exterior. Thus, only the flow direction needs to be switched, for instance under the influence of a pump.

At least one of the group consisting of the lens material removal unit, the cannula, and the further cannula may be formed so as to be aligned essentially parallel to the optical fiber, particularly so as to circumferentially surround the optical fiber, coaxially surround the optical fiber or be aligned aside the optical fiber. Taking such a measure may allow for a simple and rugged configuration of the entire device. For instance, the optical fiber may be provided as a tubular member having, for instance, a circular cross-section. Surrounded by this circular cross-section, a hollow cylindrical portion may be provided and arranged coaxially with the optical fiber. This hollow cylinder may be used as a first cannula for inserting fluid into the eye or for removing material from the eye. One or more further such hollow cylindrical cannulae may be provided surrounding the first cannula, so that the different hollow cylinders may be provided concentrically and may be surrounded by one another.

Alternatively, it is also possible to provide the different channels in parallel to one another as some kind of bundle with parallel channels.

The lens epithelial cell deactivation unit may comprise an end portion so as to enable a spatial control of the deactivating of lens epithelial cells of an eye. Such an end portion may be a longitudinal movable, rotatable and/or tiltable end portion. A diffuser (for instance spherical- or honeycomb-shaped) may be used for the UV-light to generate quasi isotropic radiation distributions (for example an abraded sphere of a fused silica material which distributes radiation). By providing the end portion of the epithelial cell deactivation unit in a flexible manner, it is possible, by adjusting the angular or longitudinal position of the end portion, the characteristics and the transmission direction of the electromagnetic radiation for selectively killing the lens epithelial cells. For instance, sensitive portions of the eye, like the retina, may be prevented from being deteriorated by electromagnetic radiation, since the movable tip may accurately define the electromagnetic radiation propagation path. The actual position of the movable tip may be controlled selectively by the physician operating the device.

The end portion may be adapted to be adjustable so as to form one of a plurality of different patterns. In other words, the radiation pattern of the electromagnetic radiation emitted at the tip may be controlled or regulated. Focusing and/or defocusing optical elements may be provided at the tip so as to have the possibility to adjust the emission characteristics in a refined manner.

Furthermore, a barrier unit may be provided and may be adapted as a mechanical barrier to an anterior chamber of an eye. Such a barrier unit, like a collar, may improve the safety when using the device and may prevent deteriorations of the human eye functionality or injuries.

The device may comprise a handle unit adapted for handling the device during operation. Such a handle unit may be a joystick which may be operated by a physician so as to control a motor moving the optical fiber and the other components. However, such a handle unit may also be directly provided as a grip or the like on the cannula surrounding the optical fiber so as to allow for a manual control of the device.

The device may comprise a user interface adapted for controlling the device in a user-controlled manner. The user interface may include a graphical user interface (for instance having a liquid crystal display, a plasma device or a cathode ray tube). Furthermore, input elements may be provided like a keypad, buttons, a touch screen, a joystick, a trackball, a foot switch, or even a microphone of a voice recognition system. Such a user interface may allow for a unidirectional or bidirectional communication of the user with the device. Fluid flow properties, light properties or a suction operation mode may be adjusted using such a user interface. Communication between the user interface and the entire apparatus may be provided in a wired manner or in a wireless manner. Control of the device is possible also from a remote location, so that it may be possible to perform a surgery procedure for instance over the Internet.

The user interface may particularly be adapted for controlling the device in at least one of the group consisting of a voice control, a foot control, a manual control, a computer display based method, and a button based control method. Particularly a foot control may allow a physician to have her or his hands free to perform the surgery, while controlling the entire system with the foot or feet.

Moreover, the device may comprise an inflatable unit adapted to be inflated in an at least partially emptied lens capsule of an eye. For recovering the natural geometry of a lens from which the lens material has been removed, such an inflatable unit may be provided which may be inflated so as to support the inner walls of the lens capsule. Therefore, for the lens epithelial cell deactivation, a shape of the lens may be provided which is close to the natural shape. This may make it dispensable to fill fluid into the lens during the application of an UV beam to the lens epithelial cells, which might, in an undesired manner, absorb a part of the electromagnetic radiation which is foreseen for deactivating the lens epithelial cells.

Furthermore, such an inflatable unit may have electromagnetic radiation absorbing properties so as to prevent a propagation of electromagnetic radiation from an inside of the lens capsule to surrounding portions, like the retina.

Such an inflatable unit may be at least one of the group consisting of an inflatable balloon, an inflatable cage, and an inflatable web.

In the following, further exemplary embodiments of the medical method will be explained. However, these embodiments also apply to the medical device.

The medical method may comprise supplying a lens capsule of an eye with a fluid, particularly with a saline solution, more particularly with a physiological saline solution. Such fillings may be particularly suitable for filling the emptied lens capsule, since the absorption characteristics of a physiological saline is appropriate, and such materials do not harm the physiological function of the eye.

The medical method may further comprise deactivating lens epithelial cells of an eye so as to prevent a secondary cataract. This may be performed in order to prevent a separate surgery of patients suffering from a second cataract after a first cataract surgery has been carried out.

The medical method may comprise deactivating the lens epithelial cells of an eye during or directly succeeding a lens body cataract surgical procedure. Thus, the first cataract surgery procedure and the second cataract surgery procedure may be combined to a single surgery procedure.

Embodiments of the invention relate to the correction of cataracts, which is a leading cause of blindness in humans. More particularly, embodiments of the invention relate to the surgical procedure known as cataract surgery or lens replacement, and related surgical methods for performing such surgery to affect a surgical cure of cataracts and fix large refractive errors. Embodiments of the invention specifically relate to surgical devices for performing eye surgery to prevent Secondary Cataracts; lens epithelial cell proliferation of the human lens. The device may be designed to kill, lyse or inhibit the human lens epithelial cells that are necessarily left behind after cataract surgery and prevent them from growing and proliferating. The growth and proliferation of these cells obscures the vision of the patient and makes a second surgery necessary. This second surgery adds risk of blindness, time and effort for the patient and financial loss for the health care system. This current second surgery technique depends on an expensive laser that is not available everywhere. Furthermore, many modern lenses, designed to accommodate or have multiple focal points depend for their proper function upon a stable, inert lens capsule. The growth of lens epithelial cells can scar and distort the shape and flexibility of the capsule and reduce the function and vision with these modern lenses.

According to an exemplary embodiment of the invention, a device and a technique are provided that may permanently prevent secondary cataracts and the related problems. The device may be able to precisely control the application of light, including but not limited to ultraviolet wavelengths, in the desired location for the desired time, wavelength and intensity. The device may include a vacuum or suction line to aid in removal and injection of fluids and gases such as air. Minimal additional risk or time will be added to the surgery and the costs and convenience to the patient will be greatly improved. A currently common second surgery with all the additional risks, costs and related burdens, may be avoided.

According to an exemplary embodiment of the invention, a device and a technique to be used in cataract surgery are provided. The device may comprise of one or several hand pieces and a control unit. The hand pieces of the device may contain together or separate cannulae. The cannulae may irrigate fluids and aspirate them as well. Air may also be injected into the eye from the hand pieces. The cannulae may contain a optical fiber that may project light into the eye in a variety or patterns. The intensity, wavelength (including but not limited to UV) and duration of the light may be controlled by the device. A footswitch, voice activation or control panel may be used to set the device. A collar may be provided on one or several of the hand pieces to separate the lens capsule from the anterior section of the eye.

The application of the light may kill or inhibit lens epithelial cells so that they do not proliferate after surgery. After the application of the light, including but not limited to UV, the capsule will likely not haze over and the patient will likely not have to undergo more surgery and be exposed to more risk.

The aspects defined above and further aspects of the invention are apparent from the examples of embodiment to be described hereinafter and are explained with reference to these examples of embodiment.

The invention will be described in more detail hereinafter with reference to examples of embodiment but to which the invention is not limited.

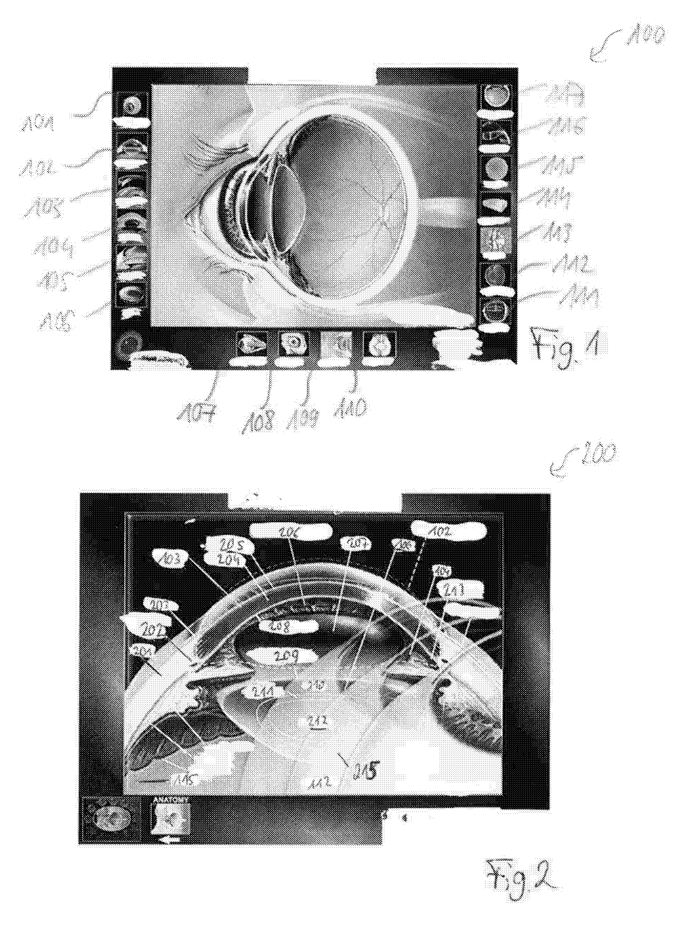

FIG. 1 is a schematic illustration of the eye.

FIG. 2 is detailed structure of the front part of the eye, the anterior segment.

FIG. 3 is a schematic illustration of a cataract removal and IOL (intraocular lens) implantation.

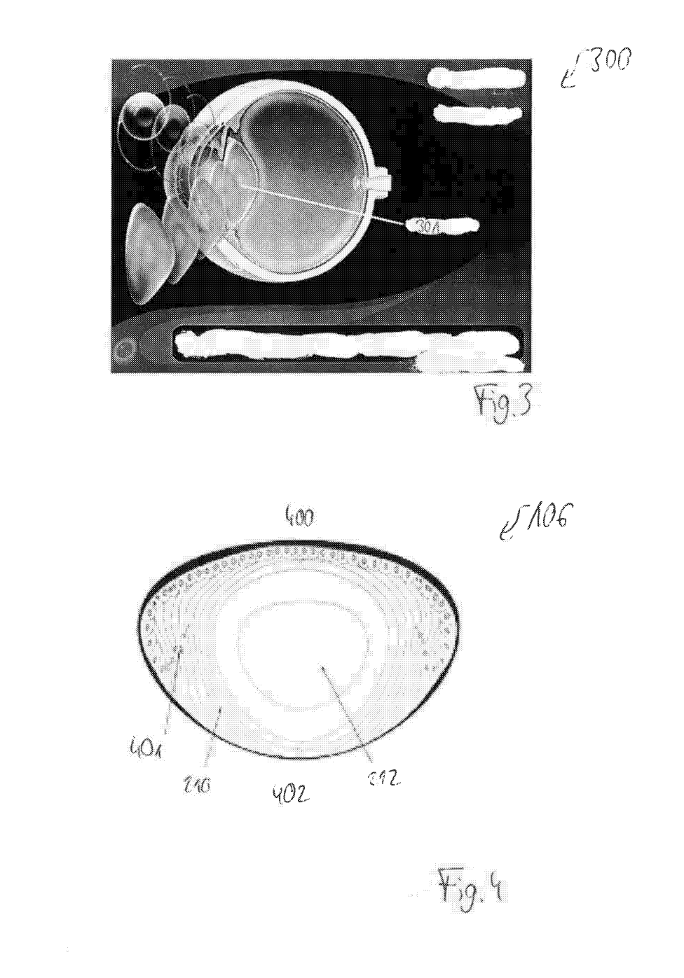

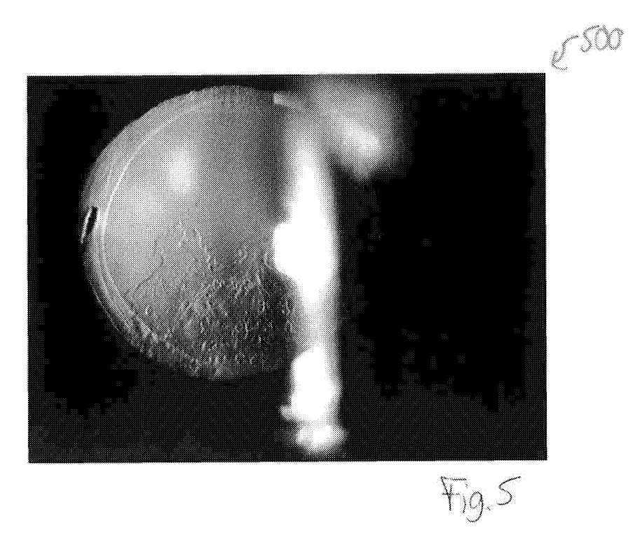

FIG. 4 is an illustration the crystalline lens horizontal cross section.

FIG. 5 an illustration of a clouded capsule from lens epithelial cells

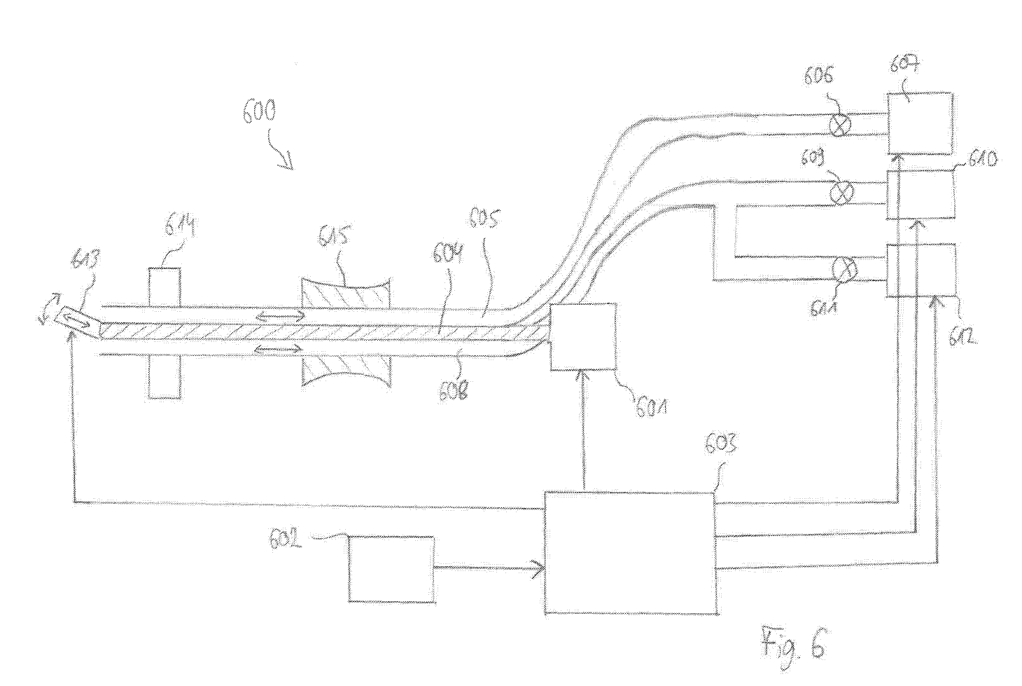

FIG. 6 is a medical device including a spherical diffuser according to an exemplary embodiment of the invention.

FIG. 7 to 9 show different optical fiber and cannula configurations of a medical device according to exemplary embodiments of the invention.

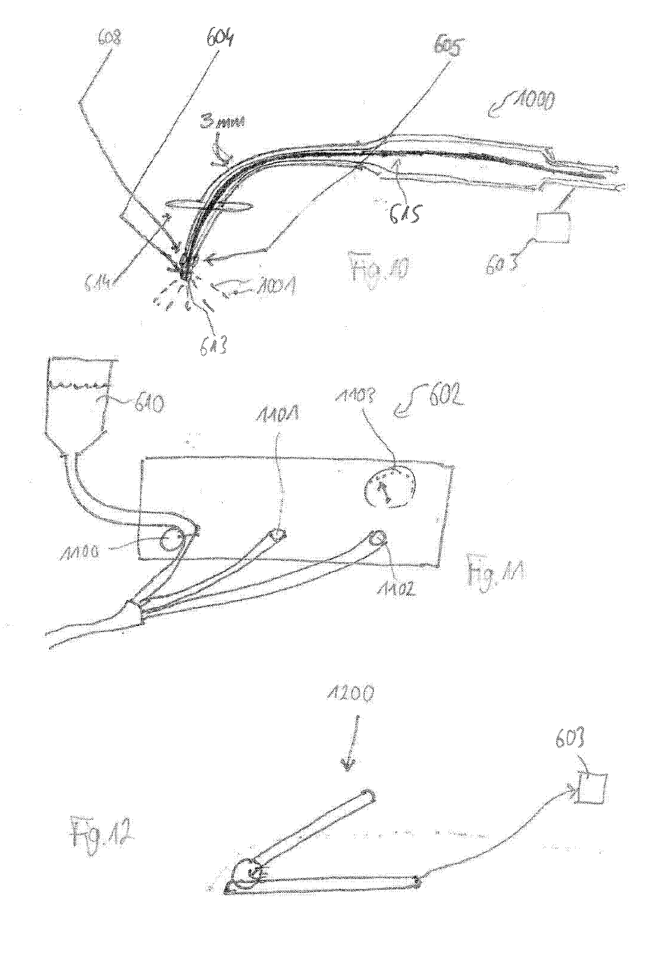

FIG. 10 is a hand piece of a medical device according to an exemplary embodiment of the invention.

FIG. 11 is a control unit of a medical device according to an exemplary embodiment of the invention.

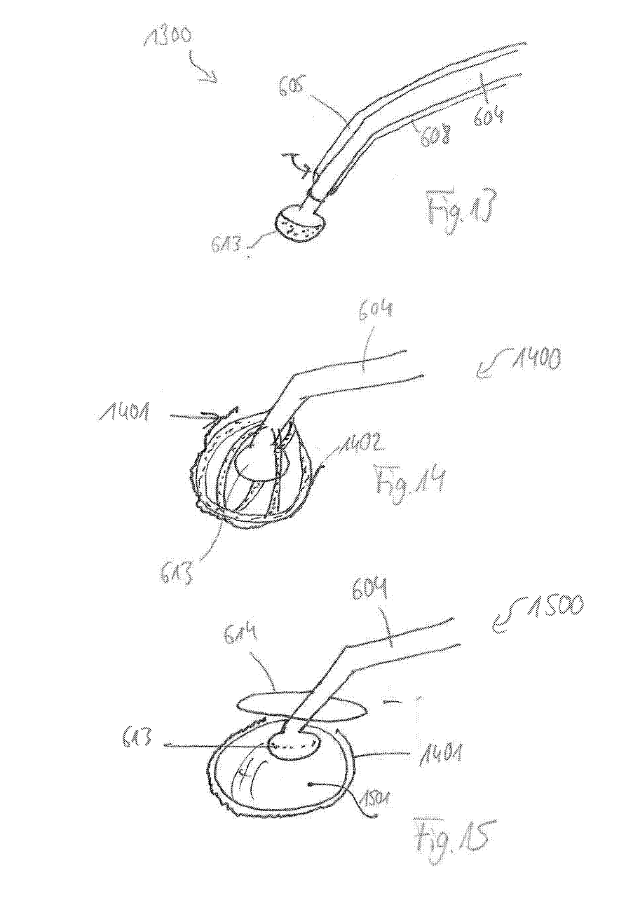

FIG. 12 is a foot switch of a medical device according to an exemplary embodiment of the invention.

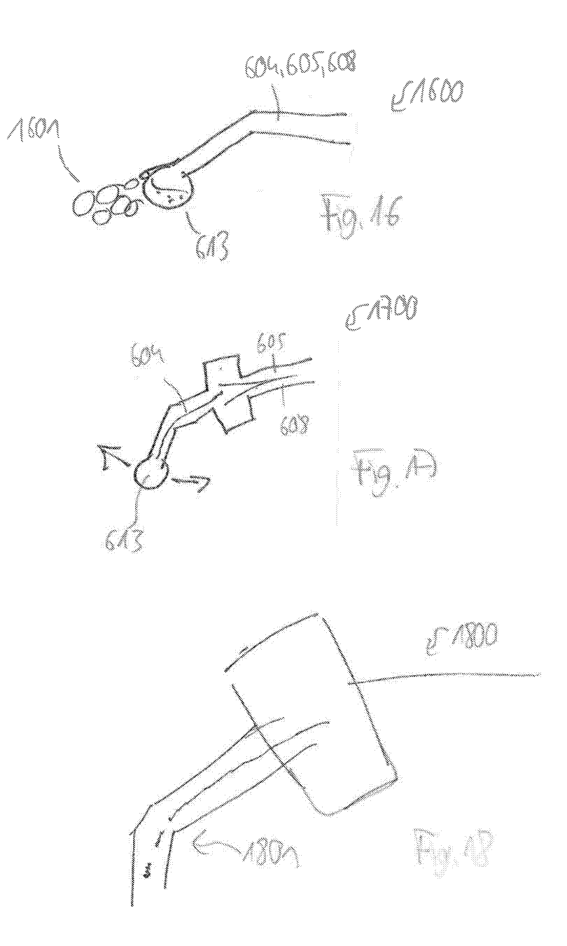

FIG. 13 is a light applicator of a medical device according to an exemplary embodiment of the invention.

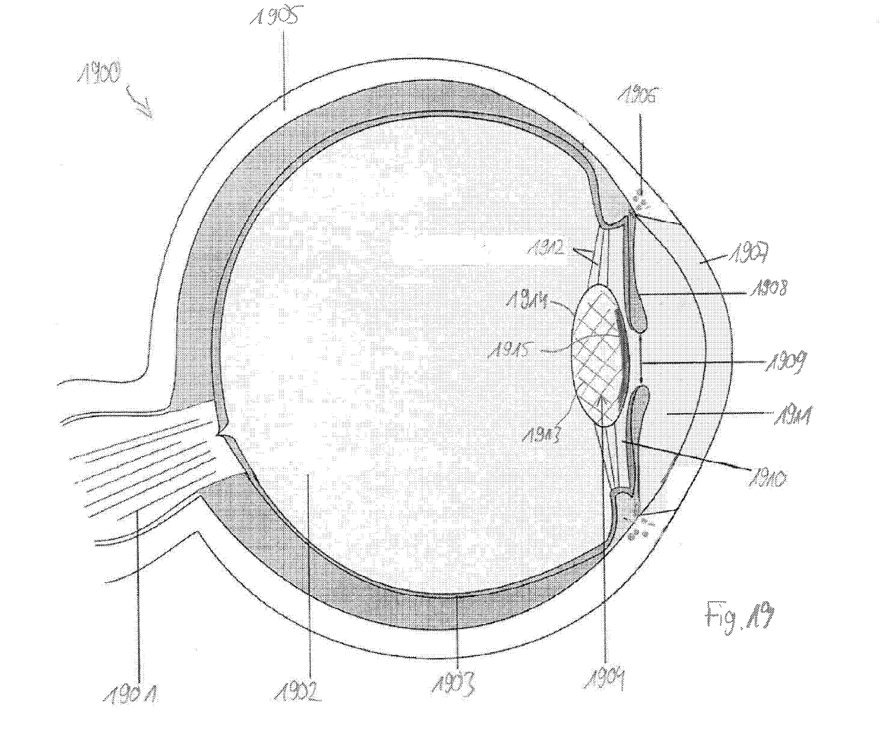

FIG. 14 and FIG. 15 show an injector with balloon and cage of a medical device according to an exemplary embodiment of the invention.

FIG. 16 is a tip and gas jet of a medical device according to an exemplary embodiment of the invention.

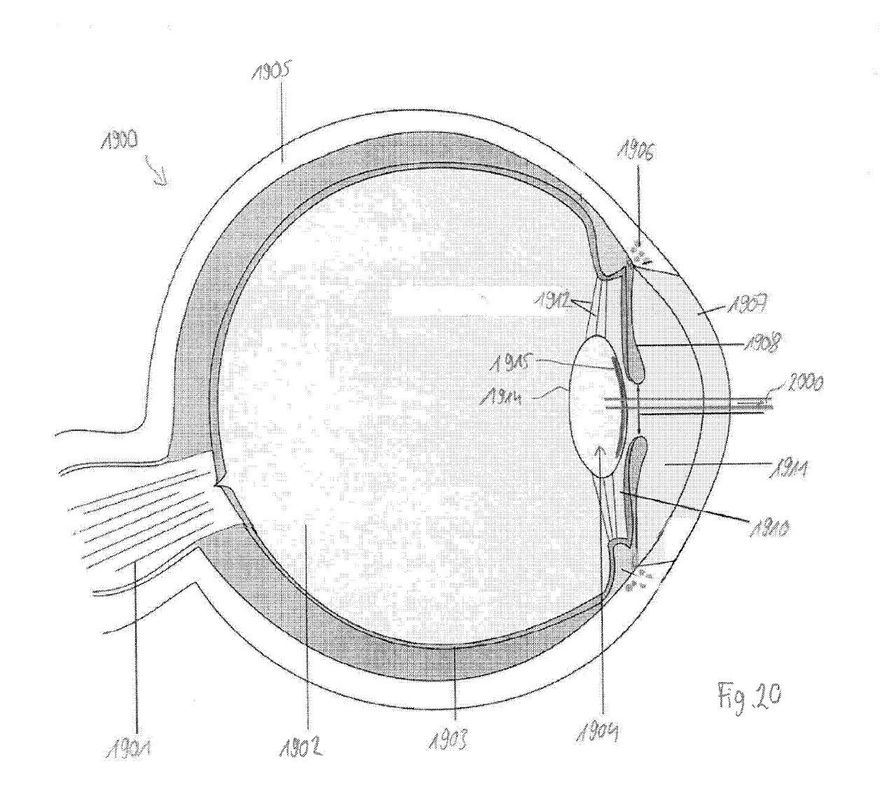

FIG. 17 is a tip with a mechanism to tilt, angle and move of a medical device according to an exemplary embodiment of the invention.

FIG. 18 is a micro manipulator of a medical device according to an exemplary embodiment of the invention.

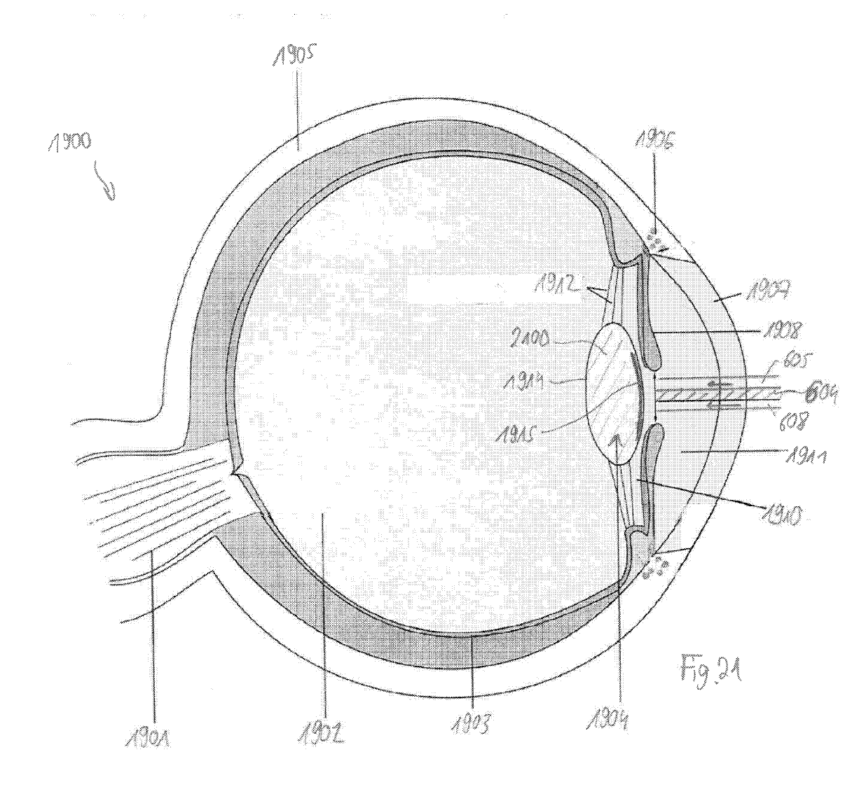

FIG. 19 to 23 show different operation modes during use of a medical device according to exemplary embodiments of the invention.

The illustration in the drawing is schematically. In different drawings, similar or identical elements are provided with the same reference signs.

Devices, techniques and methods for prevention of lens epithelial cell growth in cataract surgery according to exemplary embodiments of the invention will be explained in the following.

Exemplary embodiments of the invention relate to a technique and related devices for performing eye surgery to correct cataracts and refractive errors. More particularly, exemplary embodiments of the invention relate to the surgical removal of cataracts and the prevention of secondary cataracts. Secondary cataract is very common and causes loss of the vision after normal, conventional cataract surgery. Secondary Cataracts result from the proliferation of human lens epithelial cells that necessarily remain after cataract surgery and cause vision loss and distortion of the structures inside the eye.

The eye works on a principle very similar to that of a camera, see FIG. 1.

FIG. 1 illustrates different parts of the human eye 100, particularly a section 101, an anterior segment 102, a cornea 103, an iris 104, an angle 105, a lens 106, muscles 107, an orbit 108, a lid 109, a vision portion 110, a choroid 111, a vitreous 112, a nerve 113, a macula 114, a retina 115, a ciliary 116, and a posterior segment 117.

The iris 104 or colored portion of the eye about the pupil, functions like a shutter to regulate the amount of light admitted to the interior of the eye. The cornea 103 or clear window of the eye, and the lens 106, which is located behind the pupil, serve to focus the light rays from an object being viewed onto the retina 115 at the back of the eye.

The cornea 103 is composed of five layers. First the epithelium that is five cells thick and is usually around 60 microns thick. A thin membrane called Bowman's membrane underlies the epithelium. The mass of the cornea 103 is called the stroma, which is about 480 microns thick. The fourth layer is another, stronger but very thin membrane called Descemet's. The final layer is the endothelium, which is only one cell thick. Bowman's, Descemet's and the endothelium do not contribute significantly to the total cornea thickness. The total thickness of the cornea 103 averages around 540 microns. Once the cornea 103 and lens 106 focus the rays of light on the retina 115, the retina 115 then transmits the image of the object viewed to the brain via the optic nerve 113. Normally, these light rays will be focused exactly on the retina 115, which permits the distant object to be seen distinctly and clearly. Deviations from the normal shape of the corneal surface, however, produce errors of refraction in the visual process so that the eye becomes unable to focus the image of the distant object on the retina 115.

Hyperopia or “farsightedness” is an error of refraction in which the light rays from a distant object are brought to focus at a point behind the retina 115. Myopia or “nearsightedness” is an error of refraction in which the light rays from a distant object are brought to focus in front of the retina 115, such that when the rays reach the retina 115, they become divergent, forming a circle of diffusion and consequently, a blurred image.

The lens 106 of the eye is the other main focusing element of the eye. This lens 106 also is able to accommodate. Accommodation is the changing of the focal power of the eye so that the human eye can focus at near and far. This ability of accommodation is more pronounced in the younger eye.

As the eye ages this ability may be lost. When the typical eye reaches 40 years of age, then the lens loses its ability to focus at near. This is due to aging changes in the lens and the structure of the eye.

FIG. 2 is an illustration of a front part 200 of the human eye and illustrates further details.

FIG. 2 illustrates the front part or anterior segment, of the eye. The relationship of the lens and the anterior segment is shown.

Particularly, FIG. 2 illustrates a sclera 201, Schlemm's channel 202, a limbus 203, a stroma 204, an epithelium 205, an anterior chamber 206, a pupil 207, an endothelium 208, an anterior capsule 209, a cortex 210, an epinucleus 211, a nucleus 212, a trabecular meshwork 213, a conjunctiva, and a posterior capsule 215.

The human lens 106 rests behind the iris 104 of the eye so that the pupil 207 is centered over lens 106. The lens 106 is not a perfect sphere but is more of a slightly flattened globe or ovoid. The lens 106 is suspended in the eye behind the iris 104 by thin filaments called zonules. The zonules are connected to a circular muscle called the ciliary body 116.

When the brain wishes to see an object at near, the muscle 107 moves and the lens 106 is refocused. This is the process of accommodation. Behind the lens 106 is the vitreous body 112, which is a clear, jelly like material that fills the posterior chamber of the eye.

The human lens layers are similar in cross section to a grape. There is a capsule around the lens 106 much like the skin of the grape. There are three layers to the lens 106 that are enclosed by the capsule. The anterior portion of the capsule, just behind the pupil 207 is called the anterior capsule 209. The lens epithelial cells that line the interior aspect of the capsule. These cells are active and are responsible for the production of lens material. The most active concentration of lens epithelial cells is at the equatorial region of the lens 106. Next, as one moves into the lens 106, is the lens cortex 210. This is a softer layer that encloses the last, central core of the lens which is the nucleus 212. The nucleus 212 is the harder center of the lens 106 that becomes darker and harder with age as a cataract develops. The final layer of capsule is called the posterior capsule 215. This capsular layer is thus between the lens contents and the vitreous body 112.

An illustration 300 of a position at which a cataract 301 (for instance due to age, injury or disease) may occur is shown in FIG. 3.

FIG. 3 is an illustration of a cataract 301. The lens 106 becomes clouded in a cataract and blocks the transmission of light to the retina 115, blocking vision.

A detailed illustration of the lens 106 is presented in FIG. 4.

FIG. 4 particularly illustrates an anterior portion 400 and a posterior portion 402 of the lens 106. A Bow region 401 is illustrated as well.

Modern cataract surgery consists of removal of the cataract and insertion of a replacement, artificial lens. The human lens 106 is a major focusing element of the eye. When the human lens 106 is removed, some optical element must be introduced to replace the lost focusing power. The most common way today is by the placement of an artificial lens, typically made of silicone or acrylic material. In cataract surgery an opening is first fashioned in the anterior part of the anterior capsule 209. This technique of creating an opening is called the capsulorhexis. The opening in the anterior capsule 209 is called the Anterior Capsulotomy. The anterior capsulotomy is usually about 5 or 6 mm in diameter and centered on the pupil 207 at the anterior capsule 209. After the capsulotomy is created, the lens material, the cortex 210 and the nucleus 212 are hollowed out and removed. Typically an ultrasound device, the phacoemulsifier, is used to remove the lens contents. The ultrasound suction needle vibrates at a very high frequency to remove the cortex 210 and the nucleus 212. A viscous material is usually injected into the eye at this point in the surgery to open the capsular bag and maintain the shape of the eye for the insertion of an artificial lens. This artificial lens is typically placed into the hollow capsular bag where the lens contents were before surgery.

The thin layer of lens epithelial cells as well as lens epithelial cells in the volume is necessarily left behind when the lens material is removed from the capsular bag. These cells can then continue to grow and replicate. In many cases they grow across the posterior, intact surface of the lens capsule. When they do they are often not clear and can obstruct the vision or passage of light through the eye. When this occurs it blocks the vision all over again for the patient just like the first cataract. This condition is called “Secondary Cataract”. Furthermore, the growth of the lens epithelial cells can also result in scarring and fibrosis that can distort the shape of the capsular bag. This may cause problems with the implanted artificial intraocular lens, especially with modern accommodating lens such as the crystalens and the multifocal lens such as the Restor and Rezoom lenses. These lenses depend on the stability of the lens capsule in which they rest. A crystalens, for example, is designed to move within the capsular bag in a stable range of focusing effect. The multifocal lenses are also dependent on the stability of the human lens capsule, as they must remain centered to be optimally effective. Capsular fibrosis and scarring, from the growth of lens epithelial cells, can often distort the shape and position of the capsular bag and therefore of the artificial lens creating astigmatism, putting the lens out of focus and destroying the effect of the lens. As lens technology advances, more complex lens such as dual optic intraocular lenses will be even more at risk from this effect of the lens epithelial cells.

Today, typically this problem of Secondary Cataract, lens epithelial cell proliferation and capsular clouding, is dealt with after surgery. There is no satisfying solution for intraocular lens distortion caused by capsular distortion and fibrosis of the shape of the capsular bag. When the lens epithelial cells cause a clouding of the posterior capsule vision is lost for the patient much like the original growth process of their cataract. A second, separate surgical procedure is needed to make an opening in the posterior capsule so light can pass through. This second surgery requires a laser called a YAG laser (Yttrium Aluminum Garnet). The YAG laser is able to create extremely high levels of energy for extremely short periods of time. This energy can be concentrated or focused to a point that results in an explosion within the eye. When precisely focused on the lens capsule and epithelial cells an opening can be created by a series of these explosions, destroying the central portion of the posterior capsule and creating an opening in the posterior capsule and lens cells. This procedure is called a YAG Posterior Capsulotomy. A YAG Capsulotomy requires the patient to undergo another surgical procedure and associated visits and costs. In addition there are increased, additional risks with the YAG capsulotomy including increased intraocular pressure and the increased risk of retinal detachment. Both of these complications are potentially serious and can cause blindness. In many parts of the world, YAG lasers are not available. The standard treatment in these areas is a second surgery. This is commonly done by the insertion of a sharp needle into the eye to tear an opening into the posterior capsule. It is not desirable to create an opening in the posterior capsule at the time of surgery at the intact posterior blocks the more liquid vitreous from moving into the anterior portion of the eye. The intact posterior capsule at the end of the case lowers the risk for serious complications like infection and retinal detachment.

Conventionally, in case of a Secondary Cataract the patient must suffer through a second period of declining vision much like the first cataract until the laser procedure is done. There is not a good solution to correct distortion of accommodating and multifocal intraocular lenses from capsular scarring and fibrosis. This distortion can therefore reduce or totally eliminate the effect of accommodating lenses and multifocal lenses. In some cases not only is the effect lost but also severe visual distortion can occur resulting in the need to remove the intraocular lens and attempt to place another intraocular lens. Intraocular lens removal and replacement surgery carries significant risks and can result in the loss of an eye.

According to an exemplary embodiment of the invention, a solution for such a problem is to remove or destroy the lens epithelial cells during the initial cataract surgery so that they would not grow and proliferate causing capsular haze and distortion of the shape of the capsule. This may be achieved by a technique and method comprising a surgical technique and related device that destroy the human lens epithelial cells during the original cataract surgery. A quick, technically easy surgical step may be added to the cataract technique. The surgery may proceed normally during the removal of the lens material. At the end of the removal of the lens material with the current ultrasound and suction devices the eye is left filled with saline. At this point a combined multiple cannula and light fiber may be inserted into the eye through the normal cataract wound. This cannula may be able to irrigate saline into the eye to maintain the shape of the eye during the insertion. The multiple cannula may be used to suction out the saline from inside the eye and replace it with air rather than viscous material. A collar may also be placed on the cannulae to create a barrier to the anterior chamber of the eye so that any light, air or fluid can be contained. Once the anterior portion of the eye, from the cornea to the posterior capsule, is filled with air, a light fiber in the device may be turned on. This fiber may be connected to a light source that may generate a specific wavelength of light, including but not limited to the ultraviolet range. The wavelength, intensity and duration of the light may be selected to cause the death of the human lens epithelial cells. Once the application of light is done, the device may be removed. The cataract surgery may then proceed routinely with the injection of the viscous material and the insertion of the intraocular lens. The entire lens epithelial destruction process can be performed from one cannula device or alternatively, a second, additional cannula can be inserted from the smaller, second cataract incision which is commonly used today. The suction, irrigation and air carrying elements of the device can be divided between the two cannulae or all placed in one device. Extra steps may be minimized and very little time or risk is added to the surgery. The components of such a device can be supplied in kits.

As the percentage of population over the age of 60 increases in most developed countries so does the incidence of cataracts in this higher risk, older age group. A cataract is a progressive clouding of the natural crystalline human lens of the eye that obstructs the passage of light to the retina. Blindness can occur when the lens becomes clouded.

In fact, the leading cause of blindness today is cataract. There is a second group of younger patients who are undergoing removal of the lens for refractive surgery. These patients typically have very high refractive or focusing errors that cannot be treated with any other technique. The treatment for cataracts or such high refractive errors is surgical removal of the lens from the lens capsule. This is commonly accomplished with phacoemulsification leaving only the lens capsule. Once the human lens is removed, a main focusing element is now missing and an insertion of an intraocular lens is commonly done. In the United States alone well over 1.5 million people have cataract surgery every year. This number is going up as the Baby Boomers mature. The vast majority of these patients have an intraocular lens implanted to allow them to clearly see again. These patients are at risk for opacification of the posterior capsule by proliferation of lens epithelial cells, “secondary cataract” and a second loss of vision and productivity. A second surgery by YAG laser is needed but can only partially correct this problem.

Embodiments of the present invention provide a method to prevent this problem in patients.

Embodiments of the present invention are designed to satisfy a need after cataract surgery. Patients can have good results after cataract surgery but most will lose vision over time as the lens epithelial cells that are left behind after surgery grow to create a Secondary Cataract and block the vision. The patient is then required to undergo a second laser surgery to make an opening in the capsule. This adds time, expense and surgical risk for the patient. Additionally, the newer accommodating lenses that move within the capsular bag are often rendered ineffective by the distortion of the capsular bag caused by the growth of the epithelial cells.

According to an exemplary embodiment of the invention, a device and a technique are provided to destroy the cells during the removal of the cataract so that they cannot grow and cause these problems. The device may comprise one or several hand pieces that may contain cannulae that may irrigate and aspirate saline or other materials. Cannulae in the hand pieces may also be able to insert air into the eye. There may also be a light fiber in the devices to emit light of various wavelengths, duration and intensity. The light fiber may be fashioned at the distal tip to create a variety of patterns. A collar may also be placed on the cannulae to create a barrier to the anterior chamber of the eye.

The lens capsule of the eye is lined with epithelial cells. The lens is placed in the capsular bag after cataract surgery. These cells can proliferate and cause loss of vision as they block the passage of light.

FIG. 5 is an image 500 illustrating a clouded posterior capsule from lens epithelial cells.

In the following, referring to FIG. 6, a medical device 600 according to an exemplary embodiment of the invention will be explained.

The medical device 600 comprises a lens epithelial cell deactivation unit comprising multiple parts, as will be explained in the following, for selectively deactivating lens epithelial cells of an eye.

Among others, the lens epithelial cell deactivation unit comprises a laser 601 as an electromagnetic radiation source for emitting electromagnetic radiation in the ultraviolet frequency range. For deactivating lens epithelial cells of an eye, the electromagnetic radiation may be selected to be in a controllable range of ultraviolet radiation. Particularly, the electromagnetic radiation emitted by the laser 601 may be in a wavelength range between 190 nm and 230 nm.

A user control unit or user interface 602 is provided via which a human user may input all parameters or instructions defining an operation mode of the device 600. Particularly, the intensity, the wavelength and the duration of the emission of ultraviolet radiation by the laser 601 may be controlled by a user via the user interface 602.

The user interface 602 is coupled to a central control unit 603, which may be a computer, a conventionally wired electric circuit, an integrated circuit or a software based control unit. Under the control of the user interface 602, the CPU 603 controls all the operations to be performed by the device 600, particularly the control or regulation of the laser 601.

The lens epithelial cell deactivation unit further comprises a optical fiber 604 coupled to the laser 601, the optical fiber 604 being adapted for guiding the ultraviolet radiation emitted by the laser 601 to lens epithelial cells of an eye (not shown in FIG. 6).

A lens material removal unit of the device 600 may be adapted for removing lens material from an inside of a lens capsule of an eye. The lens material removal unit comprises a suction cannula 605 for removing lens material out of the lens capsule. Such a suction operation mode may be selected by opening a first valve 606 so that a pump 607 is connected via the cannula 605 to an interior of the lens capsule. Although not shown in FIG. 6, an ultrasound source may be provided for generating ultrasonic waves so as to promote the removal of lens material from an inside of a lens capsule of an eye.

Furthermore, a second cannula 608 is provided which is coupled via a second valve 609 to a saline container 610 and which may also be coupled via a third valve 611 to a further container 612. In the further container 612, any other desired rinse or clean fluid or the like may be stored. As indicated in FIG. 6, the CPU 603 also controls the pump 607, the containers 610, 612 and also the valves 606, 609, 611.

It is also possible that the second container 612 contains a gas (like a mixture of oxygen and nitrogen), so that the gas can be entered into a lens capsule when an end portion (left-hand side of FIG. 6) is inserted into the lens.

As can be taken from FIG. 6, the cannula system comprises two different cannula channels 605, 608. In the embodiment of FIG. 6, the cannula 605, the optical fiber 604 and the cannula 608 are provided parallel to one another, as some kind of bundle of transmission lines, one optical transmission line 604 and two material transmission lines 605, 608.

As can further be taken from FIG. 6, the device 600 comprises a tip 613 which, under the control of the CPU 603, may enable a spatial control of the deactivation of lens epithelial cells of the eye. As indicated by a curved arrow in FIG. 603, the tip 613 is rotatable or tiltable, and as indicated by another linear arrow, is movable also in a longitudinal manner. Thus, the spatial characteristics of the light emission at the tip 613 may be controlled accurately.

Additionally or alternatively to the rotatable or tiltable property of the tip 613, the device 600 may comprise, attached to or integrally formed with the tip 613, a diffuser element for a spatially distributing the electromagnetic radiation in an essentially isotropic manner. This may be a abraded sphere to distribute light emitted by the tip in a spatially isotropic way.

A collar 614 is provided as a mechanical barrier to an anterior chamber of the eye.

Furthermore, a handle unit 615 is provided as some kind of grip which allows a physician to manually control the position of the device 600. Alternatively, although not shown in FIG. 6, it is possible that the positioning of the device 600, particularly of the tip 613, the flexible optical fiber 604 and the cannulae 605, 608 is performed by an electric motor which may be controlled by a user via the user interface 602, wherein the components may be driven by the CPU 603.

The user interface 602 may allow for a user-controlled control of the device 600, and may allow for a voice control, foot control, manual control, computer display bases control, and a button based control.

In the following, referring to FIG. 7 to FIG. 9, different embodiments of cannula and optical fiber configurations will be explained.

FIG. 7 shows a system 700 in which the optical fiber 604 is circumferentially surrounded by a single capsule 605 which allows for a bidirectional transmission of fluids like gases or liquids. In other words, gases or liquids may be supplied from left to the right according to FIG. 7, or in the opposite direction.

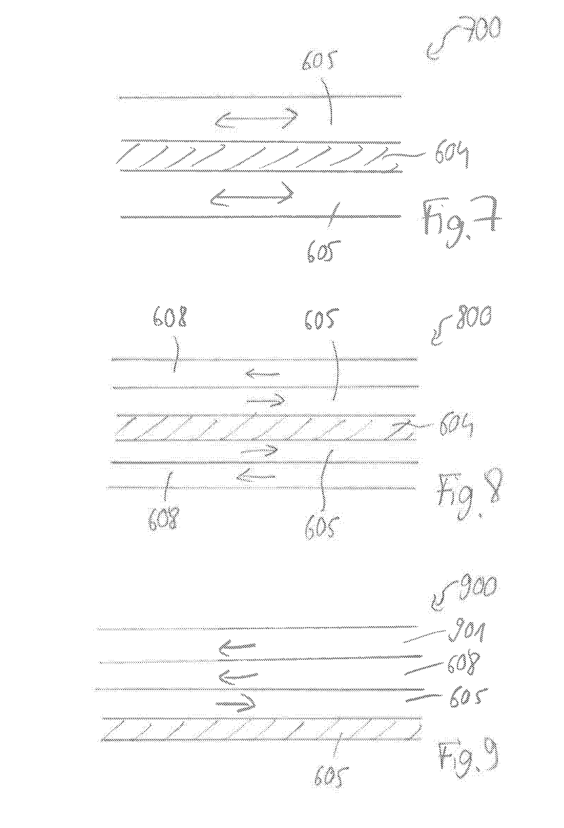

FIG. 8 shows a configuration in which the light fiber 604 is surrounded by two circumferential cannulae 605, 608, wherein the inner cannula 605 is adapted for a fluid transport from left to right, whereas the outer cannula 608 is provided for a fluid transport from right to left.

FIG. 9 shows a system 900 in which the light fiber 604, the first cannula 605, the second cannula 608 and a third cannula 901 are provided parallel to one another as some kind of bundle structure. In the embodiment shown in FIG. 9, the first cannula 605 is adapted for a material transport from left to right, whereas the two additional cannulae 608, 901 are adapted for a material transport from right to left.

FIG. 10 shows another configuration of a device 1000, more particularly of a hand piece such a device 1000.

As can be taken from FIG. 10, electromagnetic UV radiation 1001 is emitted at the tip 613 under the control of the control unit 603.

FIG. 10 illustrates a hand piece for the device 1000. The hand piece may be one or several and may contain the cannulae 605, 608 for inserting air, saline or other fluids and for suctioning and removal of fluids. The hand piece may also contain a light fiber 604 to apply light at various wavelengths, intensities and durations. The hand piece may have a collar 614 to separate the interior of the capsular bag from the anterior portion of the eye. These could be supplied in disposable kits.

FIG. 11 illustrates the user interface 602 in more detail.

Parameters like light intensity, light wavelength, light duration, and fluid flow properties may be controlled via the user interface 602. Furthermore, air injection/aspiration and fluid injection/aspiration properties may be controlled. For this purpose, among other things which are not shown in FIG. 11, a fluid on/off button 1100, a wavelength intensity light source control button 1101 and a suction button 1102 are shown. A visual inspection unit 1103 allows to control the suction parameters, like a pressure, during use of the system.

FIG. 11 is a control unit of the device 600. The control unit contains pumps to remove fluids and to insert air and/or saline into the eye. The control unit also contains a light generator to create light at various wavelengths such as ultraviolet. The intensity and duration could also be adjusted. The device 600 could be controlled by various means such as voice activation, switches on the hand piece or a footswitch.

FIG. 12 shows such a foot switch 1200 which can be controlled by a foot of a physician and can be brought to various positions correlated to corresponding functions.

The footswitch 1200 of the device 600 may control the irrigation of saline and air, aspiration of saline and an off/on operation mode for the light.

FIG. 13 is a detailed view of a light applicator 1300 of a medical device 600 according to an exemplary embodiment of the invention.

Air or gas may be supplied through the cannulae 605, 608. Light may be transmitted via the fiber optical wire 604. The system may be connected to the control unit 603, although not shown in FIG. 13. Furthermore, the fiber optics tip 613 is shown which may be controlled to spread light in a defined manner.

In the following, referring to FIG. 14, an injector 1400 with a cage 1402 will be explained.

As can be taken from FIG. 14, an end portion of the fiber 604, namely the tip 613, can be surrounded with an inflatable cage 1402. Furthermore, FIG. 14 shows a capsule 1401. This capsule 1401 can be the lens capsule to be stuffed with the inflatable cage 1402 in an inflated operation mode, or can also be an additional capsule component for shielding electromagnetic radiation to avoid a propagation of such electromagnetic radiation to especially sensitive portions of the eye, like the retina.

The inflatable cage 1402 may be inflated so as to fill the interior of the empty lens capsule so as to recover its natural shape. Then, electromagnetic radiation may be emitted by the tip 613 so as to selectively remove lens epithelial cells in interior of the lens capsule, whereas other portions of the eye may be prevented from being deteriorated. The shape recovery function of the cage 1402 may make it dispensable to fill the lens capsule with a liquid during the surgery step.

FIG. 15 shows a configuration of an injector 1500 with a balloon 1501.

In a similar manner as in FIG. 14, the configuration of FIG. 15 allows to inflate the balloon 1501 against the capsule 1401 so as to recover a shape of an internal lens capsule. The applicator 604 may be connected as a control unit 603, although not shown in FIG. 15. The balloon 1501 may be inflated with respect to the capsule 1401.

The shield may be above or below the iris, the capsule or both.

FIG. 16 is an illustration 1600 of the tip and gas jet.

At the applicator tip 613, bubbles 1601 may be generated. The cannulae 605, 608 and/or the light fiber 604 may be provided linearly or coaxially, and the cannulae 605, 608 may be used for transporting gas, for transporting liquids or for suction.

FIG. 17 shows a tilt, angle and move mechanism 1700 for tilting, angularly moving or longitudinally moving the tip 613.

FIG. 18 shows a micromanipulator 1800 with a flexible cable 1801.

In the following, referring to FIG. 19 to FIG. 23, different operation modes during use of a medical device 600 according to an exemplary embodiment of the invention will be explained.

FIG. 19 shows the human eye 1900.

The eye 1900 includes the nerve 1901, the eye body 1902, the retina 1903, the lens 1904, the sclera 1905, Schlemm's channel 1906, the cornea 1907, the iris 1908, the pupil 1909, the anterior eye chamber 1910, the front eye chamber 1911, and the eye muscles 1912.

As can further be taken from FIG. 19, the lens 1904 is filled with lens material 1913 and comprises a lens capsule 1914. Lens epithelial cells 1915 are shown as well.

As can be taken from FIG. 20, in order to treat a patient suffering from cataract, a suction channel 2000 may be inserted into the lens 1904, and the lens material may be removed from an inside of the lens 1904 by a pressure and/or by an ultrasonic sound. As a result of such a treatment, the interior of the lens 1904 is emptied, so that the lens capsule 1914 is no longer filled with lens material. However, at a front end of the lens capsule 1914, epithelial cells 1915 remain.

Conventionally, the treatment is finished by filling an artificial lens material into the lens 1904. However, in such a scenario it may occur under undesired circumstances, that the lens epithelial cells 1915 generate new lens material so that the symptoms of the cataract come back and a second surgery procedure has to be applied.

However, according to an exemplary embodiment, such undesired effects may be prevented, as will be explained in the following.

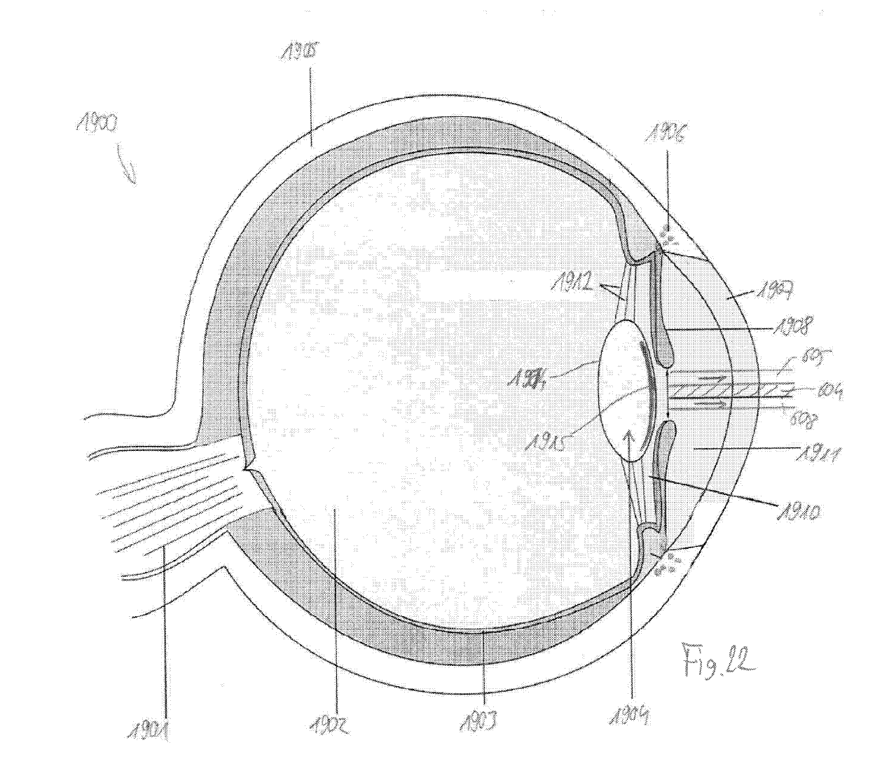

As can be taken from FIG. 21, instead of merely using a suction cannula 2000, light fiber 604 is surrounded or provided integrally formed with two cannulae 605, 608. Such a configuration may be entered into a front opening of the lens 1904, and a saline material 2100 may be injected into the empty lens capsule 1914 for cleaning purposes.

Next, as shown in FIG. 22, the cleaning fluid 2100 may be removed from the interior of the lens capsule 1914. This can be done by connecting the cannulae 605, 608 to a suction pump, which is indicated by arrows having a direction from left to right. After this, the lens capsule 1914 is empty again.

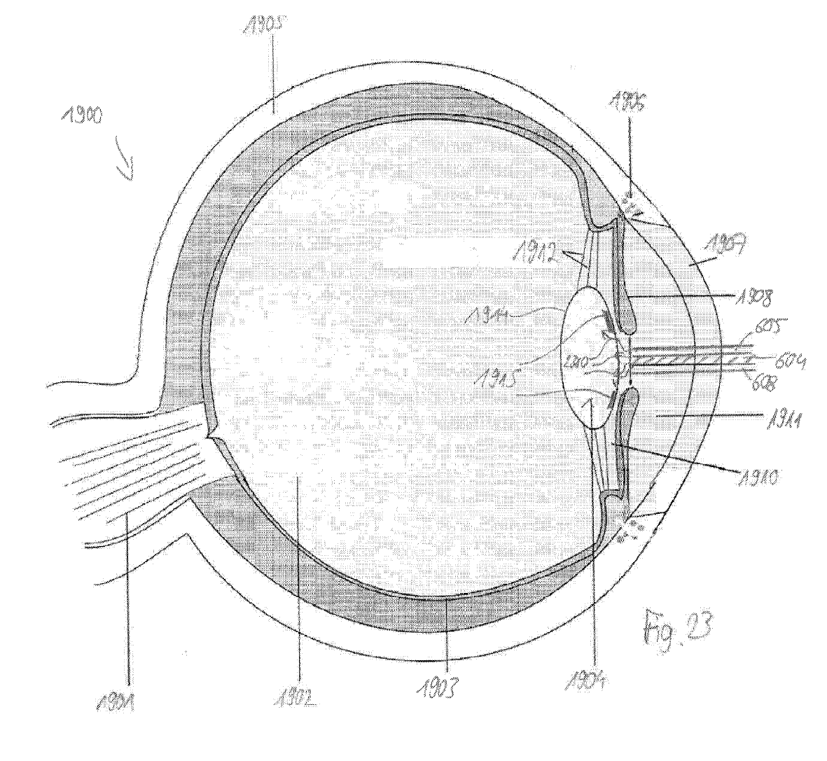

After that, as shown in FIG. 23, electromagnetic radiation 2300 in the ultraviolet wavelength range is transmitted into the interior of the empty lens capsule 1914 via the optical fiber 604. By taking this measure, at least a significant portion of the lens epithelial cells 1915 are destroyed, as shown in FIG. 23.

In order to recover the original shape or the natural shape of the lens 1904, it is possible that the device inserted into the lens capsule 1914 comprises an inflatable balloon and/or a capsule as has been described above. Furthermore, such a balloon may have absorbing functions so as to prevent the retina 1903 from being deteriorated by electromagnetic radiation 2300.

Although not shown in the figures, after the operation state of FIG. 23, a conventional cataract surgery step may be performed, namely to fill the empty lens capsule 1914 with artificial lens material.

After that, the combined first and second cataract surgery according to an exemplary embodiment of the invention is finished.

As an alternative to the configuration of FIG. 20, the suction channel 2000 may be substituted by the light fiber 604 integrally formed with two cannulae 605, 608, and the method steps described referring to FIG. 20 to FIG. 23 may be performed without changing the device interacting with the eye.

It should be noted that the term “comprising” does not exclude other elements or features and the “a” or “an” does not exclude a plurality. Also elements described in association with different embodiments may be combined.

It should also be noted that reference signs in the claims shall not be construed as limiting the scope of the claims.

1. A medical device, comprising
a lens epithelial cell deactivation unit adapted for deactivating lens epithelial cells of an eye using electromagnetic radiation.

2. The device according to claim 1,
wherein the lens epithelial cell deactivation unit comprises an electromagnetic radiation source adapted to emit electromagnetic radiation for selectively deactivating lens epithelial cells of an eye.

3. The device according to claim 2,
wherein the electromagnetic radiation source is adapted to emit ultraviolet radiation for deactivating lens epithelial cells of an eye.

4. The device according to claim 3,
wherein the electromagnetic radiation source is adapted to emit ultraviolet radiation having a wavelength of equal to or less than essentially 400 nm.

5. The device according to claim 1, wherein the electromagnetic radiation source is adapted to emit ultraviolet radiation having a wavelength of equal to or more than essentially 190 nm.

6. The device according to claim 1, wherein the electromagnetic radiation source is adapted to emit ultraviolet radiation in the range between essentially 100 nm and essentially 400 nm, particularly in the range between essentially 100 nm and essentially 400 nm, more particularly in the range between essentially 180 nm and essentially 230 nm, further particularly in the range between essentially 190 nm and essentially 220 nm, still more particularly in the range between essentially 180 nm and essentially 230 nm, still more particularly in the range between essentially 300 nm and essentially 400 nm, and still more particularly in the range between essentially 320 nm and essentially 370 nm.

7. The device according to claim 2, wherein the electromagnetic radiation source is adapted to emit electromagnetic radiation in such a manner that at least one of the group consisting of one or more wavelengths to be applied sequentially or simultaneously, an intensity, and a duration of the emission of electromagnetic radiation is controllable.

8. The device according to claim 2, wherein the lens epithelial cell deactivation unit comprises at least one guiding element of the group consisting of an optical fiber, a hollow fiber, a waveguide, and a liquid-filled fiber, wherein the at least one guiding element is coupled to the electromagnetic radiation source, and wherein the at least one guiding element is adapted for guiding electromagnetic radiation emitted by the electromagnetic radiation source to lens epithelial cells of an eye.

9. The device according to claim 1, comprising a lens material removal unit adapted for removing lens material from an inside of a lens capsule of an eye.

10. The device according to claim 9, wherein the lens material removal unit comprises a suction unit for sucking off lens material from an inside of a lens capsule of an eye.

11. The device according to claim 9, wherein the lens material removal unit comprises an ultrasound source unit for generating ultrasound so as to promote the removal of lens material from an inside of a lens capsule of an eye.

12. The device according to claim 9, wherein the lens epithelial cell deactivation unit and the lens material removal unit are integrally formed in a common device.

13. The device according to claim 1, comprising a cannula adapted to a supply a lens capsule of an eye with a fluid.

14. The device according to claim 13, wherein the cannula is adapted to supply a lens capsule of an eye with at least one of the group consisting of a liquid and a gas.

15. The device according to claim 13, wherein the cannula comprises a plurality of separate cannula channels, each of the cannula channels being adapted for separately and selectively supplying a lens capsule of an eye with a fluid.

16. The device according to claim 9, wherein the cannula and at least one of the group consisting the lens epithelial cell deactivation unit and the lens material removal unit are integrally formed in a common device.

17. The device according to claim 13, comprising a cannula control unit adapted to control a fluid flow through the cannula.

18. The device according to claim 17, wherein the cannula control unit is adapted to control at least one of a fluid flow rate and a kind of fluid to flow through the cannula.

19. The device according to claim 1, comprising a further cannula adapted to suck off a fluid from an inside of a lens capsule of an eye.

20. The device according to claim 10, wherein the further cannula and the suction unit are formed as a common device.

21. The device according to claim 13, wherein the further cannula and the cannula are formed as a common device.

22. The device according to claim 8, wherein at least one of the group consisting of the lens material removal unit, the cannula, and the further cannula is formed so as to be aligned essentially parallel to the at least one guiding element, particularly so as to circumferentially surround the at least one guiding element, coaxially surround the at least one guiding element or be aligned aside the at least one guiding element.

23. The device according to claim 1, wherein the lens epithelial cell deactivation unit comprises an end portion so as to enable a spatial control of the deactivating of lens epithelial cells of an eye.

24. The device according to claim 23, wherein the end portion is at least one of the group consisting of longitudinally movable, rotatable, and tiltable.

25. The device according to claim 23, wherein the end portion is adapted to be adjustable so as to form one of a plurality of different patterns.

26. The device according to claim 1, comprising a diffuser element for a spatially distributing the electromagnetic radiation in an essentially isotropic manner.

27. The device according to claim 1, comprising a barrier unit adapted for providing a mechanical barrier to an anterior chamber of an eye.

28. The device according to claim 27, wherein the barrier unit is adapted as a collar.

29. The device according to claim 1, comprising a handle unit adapted for handling the device during operation.

30. The device according to claim 1, comprising a user interface adapted for controlling the device in a user-controlled manner.

31. The device according to claim 30, wherein the user interface is adapted for controlling the device in at least one manner of the group consisting of a voice control, a foot control, a manual control, a computer display based control, and a button based control.

32. The device according to claim 1, comprising an inflatable unit adapted to be inflated in an at least partially emptied lens capsule of an eye.

33. The device according to claim 32, wherein the inflatable unit is at least one of the group consisting of an inflatable balloon, an inflatable cage, and an inflatable web.

34. The device according to claim 2, wherein the electromagnetic radiation source is adapted for generating the electromagnetic radiation when being located within a lens capsule of the eye.

35. The device according to claim 34, wherein the electromagnetic radiation source is adapted for inducing emission of ultraviolet radiation by igniting a plasma using a laser directly in the lens capsule.

36. A medical method, comprising deactivating lens epithelial cells of an eye using electromagnetic radiation.

37. The medical method of claim 36, comprising supplying a lens capsule of an eye with a fluid, particularly with a saline solution, more particularly with a physiological saline solution.

38. The medical method of claim 36, comprising deactivating lens epithelial cells of an eye so as to prevent a secondary cataract.

39. The medical method of claim 36, comprising deactivating lens epithelial cells of an eye during or directly succeeding a lens body cataract surgical procedure.

40. The medical method of claim 36, comprising deactivating lens epithelial cells of an eye for treating cataract by surgery.

41. A medical device, comprising a lens epithelial cell deactivation unit adapted for deactivating lens epithelial cells of an eye by irradiating the epithelial cells with ultraviolet electromagnetic radiation, and a lens material removal unit adapted for removing lens material from an inside of a lens capsule of an eye.

42. A medical device, comprising:
a lens epithelial cell deactivation unit adapted for deactivating lens epithelial cells of an eye by irradiating the epithelial cells by electromagnetic radiation having a wavelength between 180 nm and 230 nm.

43. A medical method, comprising:
deactivating lens epithelial cells of an eye by irradiating the epithelial cells with electromagnetic radiation having a wavelength between 180 nm and 230 nm.
